(19) United States
(12) Patent Application Publication
(10) Pub. No.: US 2022/0133963 A1
(43) Pub. Date: May 5, 2022

(54) PERSONAL ACCESSORY KIT FOR A BREAST PUMP AND ACCOMPANYING TUBING ADAPTER

(71) Applicants: John Conklin, Carlsbad, CA (US); Sen-Yuan Ro, Carlsbad, CA (US)

(72) Inventors: John Conklin, Carlsbad, CA (US); Sen-Yuan Ro, Carlsbad, CA (US)

(21) Appl. No.: 17/508,926

(22) Filed: Oct. 22, 2021

Related U.S. Application Data
(60) Provisional application No. 63/108,300, filed on Oct. 31, 2020.

(51) Int. Cl. A61M 1/06; A61M 39/10

(52) U.S. Cl. CPC A61M 1/062; A61M 2039/1077; A61M 1/064; A61M 39/10

(57) ABSTRACT

A personal accessory kit (PAS) for a breast pump is provided. The PAS and breast pump together form a complete breast pump system. The PAS has a locking mechanism to lock a diaphragm base of the system to a cap of the diaphragm of the system. The PAS further has an ergonomically-shaped handle. A flattened port adapter plug is provided for reducing single pumping vacuum pressure when a single port of the breast pump is used. The adapter allows for the safe utilization of a single port of a breast pump.

### REFERENCE TO RELATED APPLICATIONS

This application claims priority to U.S. Provisional Application No. 63/018,300 which was filed on Oct. 31, 2020, the entire contents of which are incorporated by reference herein.

### BACKGROUND OF THE INVENTION

A personal accessory kit (PAS) for a breast pump is provided. The PAS and breast pump together form a complete breast pump system. The PAS has a locking mechanism to lock a diaphragm base of the system to a cap of the diaphragm of the system. The PAS further has an ergonomically-shaped handle. A flattened port adapter plug is provided for reducing single pumping vacuum pressure when a single port of the breast pump is used. The adapter allows for the safe utilization of a single port of a breast pump.

Breast pumps are common. For example, U.S. Pat. No. 10,744,243 to Rigert discloses a breast pump for pumping human breast milk having a flexible pumping membrane and a stiff cap, which can be fitted onto said pumping membrane, this resulting in the formation of a pumping chamber for generating a negative pressure. The cap can be, or is, connected to a breast-shield unit via a suction line. The pumping membrane has an encircling collar with a step and a laterally projecting protrusion. The cap has, on its inner side, an encircling abutment region and also at least one form-fitting element, which is spaced apart from the encircling abutment region and establishes a form fit with the protrusion of the pumping membrane. The cap is positioned with a snap fit on the pumping membrane. In the abutment region (53), it establishes a joint frictional fit with a step of the pumping membrane and thus forms a seal. The snap-fit positioning of the cap on the pumping membrane is sufficient for the cap to be retained with sealing action on the pumping membrane.

Further, U.S. Pat. No. 10,568,996 to Aalders discloses a breast pump for extracting milk from a human breast having a first breast receiving funnel for receiving a first breast of a user; a vacuum pump for generating an underpressure within the first breast receiving funnel; and a control unit for controlling the vacuum pump; wherein the control unit is configured to operate the vacuum pump in at least two different modes, an attachment mode and a milk extraction mode; wherein in the attachment mode the control unit is configured to control the vacuum pump to generate a constant underpressure within the first breast receiving funnel allowing the user to attach the first breast receiving funnel to the first breast; and wherein in the milk extraction mode the control unit is configured to control the vacuum pump to generate a time-variable underpressure profile within the first breast receiving funnel for extracting milk from the first breast.

Still further, U.S. Pat. No. 10,525,176 to Makower discloses a pump for pumping milk from a breast, wherein the milk is expressed from the breast under suction and milk is expulsed from the pumping mechanism to a collection container under positive pressure.

However, these patents fail to describe a breast pump which is easy to use and efficient. Further, these patents fail to provide for a breast pump having an accompanying tubing adapter which allows a user to use a single port of a pump in a safe and efficient manner.

### SUMMARY OF THE INVENTION

A personal accessory kit (PAS) for a breast pump is provided. The PAS and breast pump together form a complete breast pump system. The PAS has a locking mechanism to lock a diaphragm base of the system to a cap of the diaphragm of the system. The PAS further has an ergonomically-shaped handle. A flattened port adapter plug is provided for reducing single pumping vacuum pressure when a single port of the breast pump is used. The adapter allows for the safe utilization of a single port of a breast pump.

An advantage of the present breast pump system is that the present breast pump system has a tube adapter for safely reducing pressure and allowing a single pump of the system to be used.

Another advantage of the present breast pump system is that the present breast pump has a locking mechanism to lock the diaphragm base of the system to a cap in a secure manner.

Still another advantage of the present breast pump system is that the present breast pump system has an ergonomically-shaped handle to allow a user to effortlessly hold the PAS and operate the pump easily.

Further, an advantage of the present breast pump system is that the present breast pump system has an ergonomically-shaped handle which reduces the chances of a user accidentally dropping the pump while using the pump.

For a more complete understanding of the above listed features and advantages of the present breast pump system reference should be made to the detailed description and the drawings. Further, additional features and advantages of the invention are described in, and will be apparent from, the detailed description of the preferred embodiments.

### BRIEF DESCRIPTION OF THE DRAWINGS

FIG. 1 illustrates a side view of the funnel portion of the pump of the system in one embodiment.

FIG. 2 illustrates a perspective view of the tube adapter having a flattened plug in one embodiment.

FIG. 3 illustrates a side view of the tube adapter having a flattened plug in one embodiment.

FIG. 4 illustrates a side view of the tube adapter having a flattened plug in one embodiment.

FIG. 5 illustrates a front view of the tube adapter having a flattened plug in one embodiment.

FIG. 6 illustrates a back view of the tube adapter having a flattened plug in one embodiment.

FIG. 7 illustrates a top view of the tube adapter having a flattened plug in one embodiment.

FIG. 8 illustrates a bottom view of the tube adapter having a flattened plug in one embodiment.

FIG. 9 illustrates the present tube adapter unit secured to the pump wherein one of the two adapter ports is sealed-off by the flattened plug.

FIG. 10 illustrates the tube adapter unit secured to the actual pump wherein both of the two adapter ports are being used and therein two funnels may be used at the same time.

FIG. 11 illustrates the tube adapter having a flattened plug inserted into one of the ports of the adapter and wherein the space created by the flattened edge of the plug is visible.

### DETAILED DESCRIPTION OF THE PREFERRED EMBODIMENTS

A personal accessory kit (PAS) for a breast pump is provided. The PAS and breast pump together form a complete breast pump system. The PAS has a locking mechanism to lock a diaphragm base of the system to a cap of the diaphragm of the system. The PAS further has an ergonomically-shaped handle. A flattened port adapter plug is provided for reducing single pumping vacuum pressure when a single port of the breast pump is used. The adapter allows for the safe utilization of a single port of a breast pump.

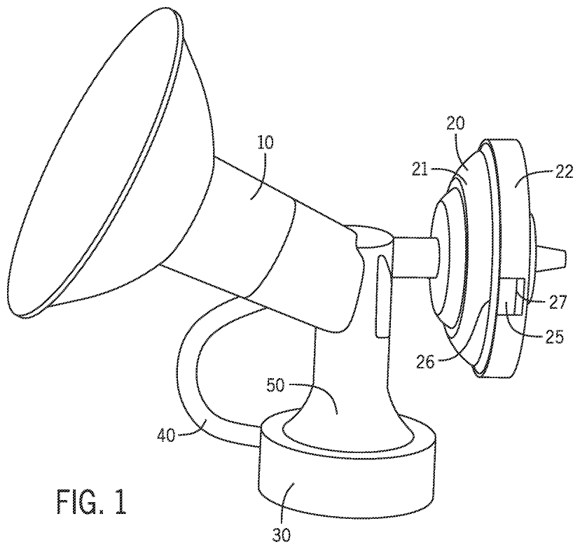

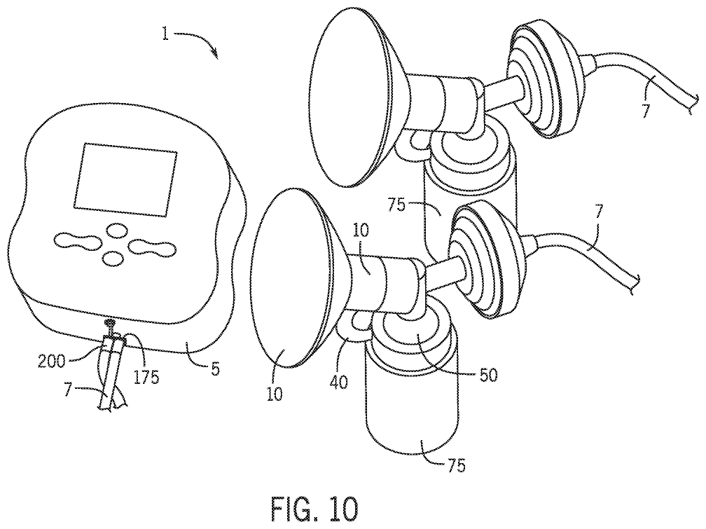

Referring first to FIG. 10, in an embodiment, a breast pump system 1 is provided. The breast pump system 1 may have a pump 5, at least one tube 7 and a detachable funnel unit 10 wherein the funnel unit 10 is secured to a diaphragm base 20. The detachable funnel 10 may have a diaphragm base 20 (FIG. 1), a flange base 30, a handle 40 and a main housing 50. The breast pump system 1 may be made of various materials including, for example, plastic, silicone and/or rubber.

The detachable funnel 10 may be the portion of the breast pump system that receives the breast. The main housing 50 may be the portion of the breast pump system that directs the milk into a storage reservoir 75 (FIG. 10). The storage reservoir 75 may be detached from the main housing 50 so that the milk may be properly used, stored or discarded.

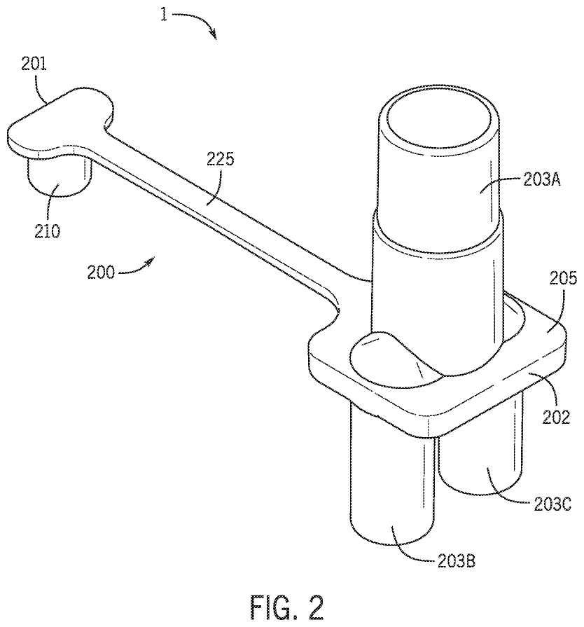

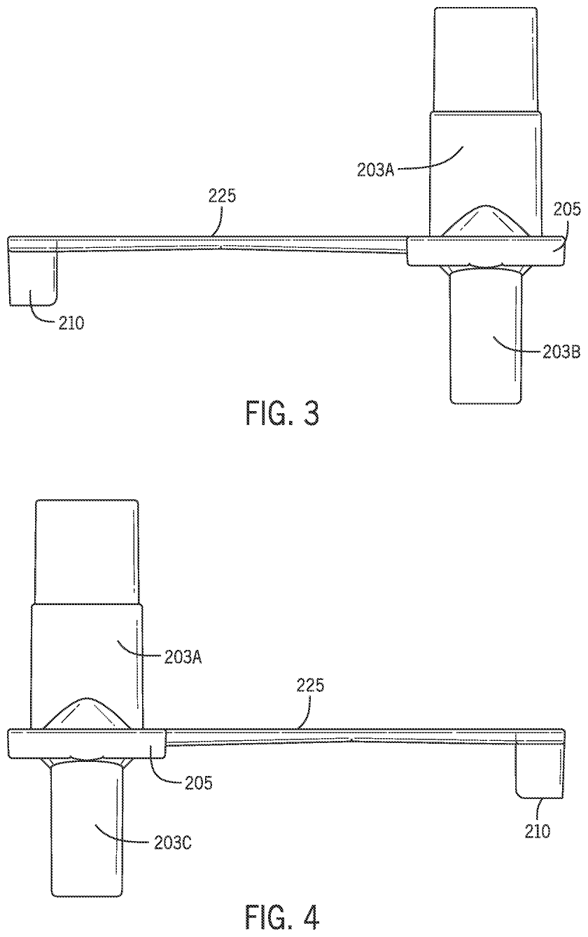

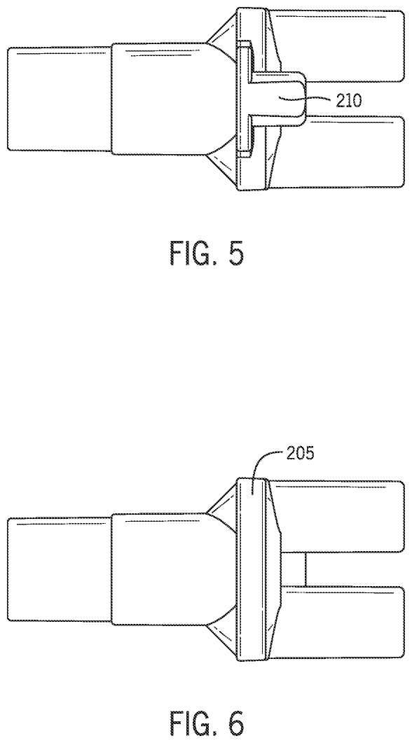

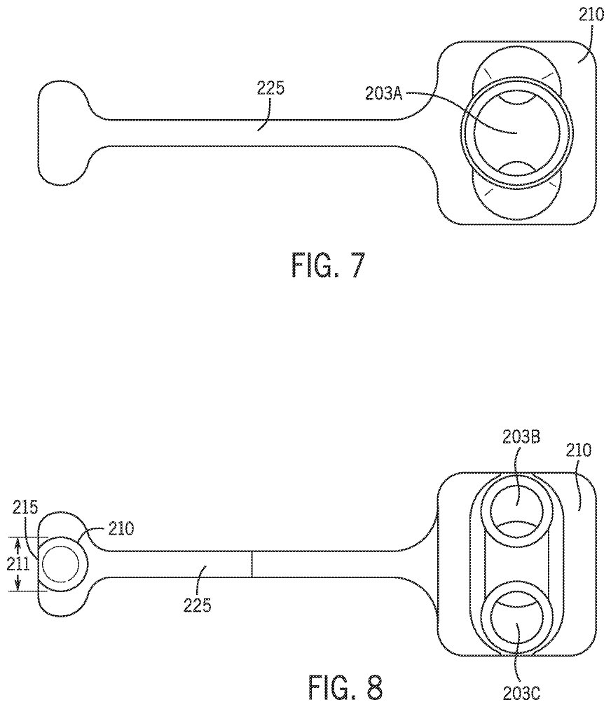

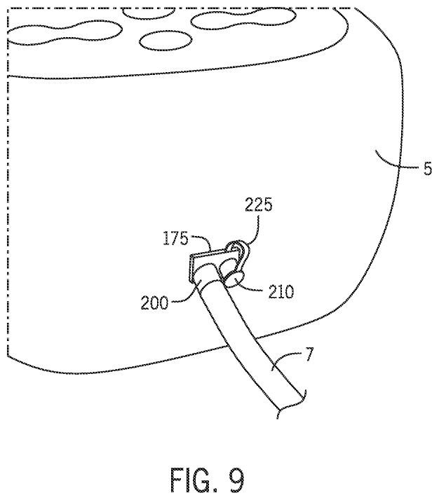

Referring now to FIGS. 2-8, in an embodiment, in an embodiment, the present PAS system 1 may have an adapter unit 200 which may alter/control the air pressure being drawn by the pump 5. The adapter unit 200 may allow a user to electively use only one of the two ports 203B, 203C of the adapter unit 200 in a safe and efficient manner (unlike prior art breast pumps). During use, the adapter unit 200 may be secured to an inlet port 175 of the pump 5 (See FIGS. 9 and 10). The inlet port 175 of the pump 5 may be the portion of the pump 5 that pulls air by creating a decrease in air pressure.

The present adapter 200 may have a first side 201 and a second side 202 wherein the first side 201 and the second side 202 are separated by a flexible bridge 225. The flexible bridge 225 may not allow the passage of air from the first side 201 to the second side 202. The second side 202 may have three “ports” 203A, 203B and 203C wherein port 203A is separated by a base 205 from port 203B and 203C. The first port 203A may be secured directly to the inlet port 175 of the pump 5 as shown in FIGS. 9 and 10. The two remaining ports 203B and 203C may receive the air tubes 7 that go to the diaphragm base 20 of the funnel 10. If a user wishes to use two funnels 10 at the same time to pump both breasts, then both of the ports 203B and 203C will each receive a different tube 7 (see FIG. 10) which each goes to a separate diaphragm 20 and a separate funnel 10. If a user only wishes to use one funnel 10, then one of the two ports 203B or 203C must be substantially sealed off by the adapter 200.

The first end 201 of the adapter 200 may have a plug 210. As best seen in FIG. 8, the plug 210 may be generally circular having a diameter 211; however, the plug 210 may have one side having a flat surface 215. In use, if the user wishes to use only one funnel 10 instead of two, then the plug 210 is inserted into one of the ports 203B or 203C so that one port 203B or 203C is substantially sealed off and the suction of the pump 1 will therein pull milk only through the other port 203B or 203C (see FIG. 9). The flat surface 215 of the plug 210 allows for the release of a small amount of air pressure from the pump 1 so that all of the pulling pressure of the pump 1 is not completely transferred to the used port 203B or 203C.

The plug 210 may have a diameter which is substantially the same as the diameter of the hollow openings of the second or third ports 203B, 203C (ignoring the flat surface 215 of the plug 210 of course). As a result, the plug 210 may be temporarily and removably secured within the opening of port 203B or 203C by friction. When the plug 210 is within the opening of one of the ports 203B or 203C, the flat surface 215 is the only area of the opening of the ports 203B or 203C which remains unblocked. Air may therefore flow from port 203B or port 203C to the first port 203A so that air may move from the pump 5 to the tube 7 while passing through the pump 5 inlet port 175 of the pump 5.

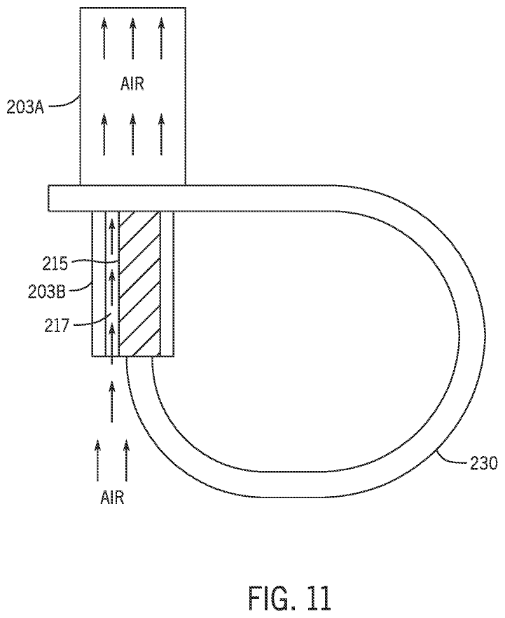

More specifically, as shown in FIG. 11 because of the flat surface 215 of the side of the plug 210 of the adapter 200, when the plug 210 is inserted into one of the ports 203B or 203C there is a small gap 217 in port 203B or 203C which allows the air pressure to be decreased, therein decreasing the overall pulling pressure from the pump 5 on the used port. The flat surface 215 of the plug 210 results in the plug 210 occupying less than the entire area of the port 203B or 203C for which it is inserted into. As a result, a small amount of air can pass through the port 203B or 203C for which the plug 210 is inserted into. FIG. 9 illustrates the adapter plug 210 inserted into one of the two ports so that only one funnel 10 is used. FIG. 10 illustrates use of two tubes 7 and, therefore, two funnels 10 to pump both breasts at the same time.

The present dual-port tubing adapter 200 has a unique plug 210 design that reduces vacuum pressure when single electric pumping is performed as opposed to when double pumping is performed. The dual-port tubing adapter 200 is a small part where the vacuum tubing 7 connects the PAS Kit to the breast pump 5. It is common for breast pump manufacturers to support double electric pumping via their breast pumps and associated accessory sets where the maximum vacuum level does not exceed safe negative pressure levels. However, it is common when a user wants/needs to perform single, electric pumping, vacuum pressure can potentially increase to unsafe (high negative pressure) levels causing personal injury as a result of prior art adapters lacking a venting mechanism as described by the present flat surface 215 of the adapter 200.

This can happen because most prior art breast pumps are designed to hit a maximum vacuum level in the most common, double pumping configuration and when one of the channels are closed/blocked with some sort of plug (typical), the vacuum pressure of the remaining open ‘channel’ increases and can increase to unsafe levels if the user is not careful with the device and its settings. The present adapter 200 only partially closes-off one of the two ports 203B, 203C to therein keep the maximum vacuum pressure, when single-pumping, to a safe level no matter what settings the user chooses.

Referring again to FIG. 1, in an embodiment, an ergonomically-shaped handle 40 is provided. The handle 40 may allow a user to hold and operate the funnel 10 with a single hand and may allow the user to operate the PAS 1 in an easy and comfortable manner. The present unique handle 40 allows a user to insert and hold the flange body and bottle with a thumb, which provides an improved user experience when using the optional manual hand pump adapter with the PAS Kit 1. The ergonomically-shaped handle 40 may allow a user to effortlessly hold the PAS and operate the pump easily. Further, the ergonomically-shaped handle 40 may reduce the chances of a user accidentally dropping the pump while using the pump.

In an embodiment, the pump system 1 may have a unique locking base associated with the diaphragm unit 20. More specifically, the diaphragm unit 20 may have a cap portion 22 and a base portion 21 which temporarily and selectively lock together by a unique locking mechanism 26. The locking mechanism 26 may be, for example, a spring-loaded flange of the cap portion 22 which temporarily grasps an extension perimeter 27 of the base portion 21. More specifically, the locking mechanism 26 may grasp a top ridge 25 of any portion of the extension perimeter 27 of the base portion 21.

The locking mechanism 26 may therefore grasp and lock onto the base portion 21 over the entire three-hundred-and-sixty-degree circumference of the base portion 21. As a result, a user is not required to precisely align the cap portion 22 with the base 21 and, instead, may simply snap the cap portion 22 onto the base portion 21 at any location of the extension perimeter 27 of the cap portion 21. The present backflow design is an impervious, circular diaphragm made out of, for example, silicone material that fits inside of the diaphragm cap 22 and diaphragm base 21. The diaphragm cap 22 may have the locking mechanism 26 to lock onto the diaphragm base portion 21 to ensure the assembly does not come apart while the product is being used.

Although embodiments of the invention are shown and described therein, it should be understood that various changes and modifications to the presently preferred embodiments will be apparent to those skilled in the art. Such changes and modifications may be made without departing from the spirit and scope of the invention and without diminishing its attendant advantages.

We claim:

1) A breast pump kit comprising:
a pump having a port;
a funnel unit having a reservoir;
at least one air tube connected between the pump and the funnel unit or reservoir;
a diaphragm connected to the funnel unit;
wherein the diaphragm has a cap portion; and
an adapter unit removably connected to the port of the pump wherein the adapter unit has a first port, a second port and a third port wherein the first port of the adapter unit is directly connected to the port of the pump and wherein the second port and the third port may each receive an air tube.

2) The breast pump kit of claim 1 further comprising:
a first end and a second end of the adapter unit wherein the first end and the second end of the adapter unit are separated by a flexible bridge.

3) The breast pump kit of claim 2 further comprising:
a base located at the second end of the adapter unit wherein the base has a top and a bottom and wherein the top of the base has the first port and wherein the second port and the third port are located on the bottom of the base.

4) The breast pump kit of claim 2 further comprising:
a plug located at the first end of the adapter unit.

5) The breast pump kit of claim 4 wherein the plug has a flat surface located on its side which creates an air passage within either the second port or the third port of the adapter unit when the plug is inserted into either the second port or the third port of the adapter unit.

6) A breast pump kit comprising:
a pump;
a funnel unit having a reservoir;
at least one tube connecting the pump to the funnel unit or reservoir;
a diaphragm connected to the funnel unit;
wherein the diaphragm has a cap portion; and
wherein the diaphragm has a spring-loaded locking mechanism to temporarily lock the diaphragm to the cap portion.

7) The breast pump kit of claim 6 wherein the diaphragm may be secured to the cap portion in a three-hundred-and-sixty-five degree manner around a circumference of the cap portion.

8) A breast pump kit comprising:
a pump;
a funnel unit having a breast receiving unit, a main housing and a reservoir;
at least one tube connecting the pump to the funnel unit or reservoir;
a diaphragm connected to the funnel unit;
wherein the diaphragm has a cap portion;
wherein the diaphragm is temporarily locked the diaphragm to the cap portion; and
a handle wherein the handle connects the main housing of the funnel unit to the breast receiving unit of the funnel.
